(19) United States
(12) Patent Application Publication
(10) Pub. No.: US 2023/0172490 A1
(43) Pub. Date: Jun. 8, 2023

(54) SYSTEM AND METHOD FOR UNSUPERVISED MONITORING IN MOBILITY RELATED DISORDERS

(71) Applicant: Kinetikos Driven Solutions, S.A., Coimbra (PT)

(72) Inventor: Ricardo da Costa Branco Ribeiro Matias, Castanheira do Ribatejo (PT)

(21) Appl. No.: 18/050,675

(22) Filed: Oct. 28, 2022

(30) Foreign Application Priority Data
Nov. 24, 2021 (PT) 117597
Aug. 19, 2022 (PT) 118164

(51) Int. Cl. A61B 5/11

(52) U.S. Cl. CPC A61B 5/112; A61B 2562/0219; A61B 5/1123

(57) ABSTRACT

Systems and methods for unsupervised monitoring of a movement related disorder experienced by a subject, the method according to one or more embodiments comprising collecting raw inertial data that represents local three dimensional (“3D”) orientation of the subject, extracting necessary inertial data from the raw inertial data, and analyzing the resultant extracted inertial data with respect to gait patterns to arrive at a gait classification on the basis of the analyzed, extracted data. The gait classification is provided as input to a recommendation process.

### CROSS-REFERENCE TO RELATED APPLICATIONS

This patent application is based on and claims priority to Portugal Provisional Patent Application, Serial Number 117597, filed Nov. 24, 2021 and entitled SYSTEM AND METHOD FOR UNSUPERVISED MONITORING IN MOBILITY RELATED DISORDERS, and further this patent application is based on and claims priority to Portugal Provisional Patent Application, Serial Number 20221000003189, filed Aug. 19, 2022, and entitled SYSTEM AND METHOD FOR UNSUPERVISED MONITORING IN MOBILITY RELATED DISORDERS, both of which are incorporated by reference, as if set forth in their respective entireties herein.

### FIELD OF THE INVENTION

The inventions described herein generally relate to unsupervised monitoring and data collection for mobility related disorders, such as Parkinson's Disease (“Parkinson's”). More specifically, inventions disclosed and described herein relate to solutions for providing valuable ecological information with regard to the severity and burden that an individual is experiencing with respect to a variety of disorders, such as Parkinson's Disease and Parkinsonism related disorders, Alzheimer's disease, Huntington's disease, Osteoarthritis, Multiple Sclerosis, etc.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND OF THE INVENTION

Neuromusculoskeletal and other mobility related disorders and impairments present a variety of impediments to the successful navigation and enjoyment of life by individuals who suffer from such conditions. For example, Parkinson's is a complex neurodegenerative disorder, with a multitude of fluctuating and heterogeneous motor and non-motor manifestations. The currently available therapeutic interventions drastically improve symptoms and quality of life of early stage Parkinson's. After several years of treatment with such therapies, individuals tend to suffer from both motor and non-motor complications, leading to a deterioration in quality of life, as well as increases in caregiver burden and healthcare resource consumption.

When identifying and tracking the progress of a given mobility related disorder, it is important to provide the clinician with an understanding of daily mobility function for an individual (e.g., performance) as a complement to the clinical assessment conducted in a standard environment (e.g., capacity). One of the building blocks of daily mobility function is the ability to walk, which enables independence and participation in social activities, as well as contributes to global health.

Understanding issues affecting the gait cycle of the individual requires a deconstruction of the process, which can be decomposed in many ways, one of which is illustrated at FIG. 1. A typical gait cycle 102 that is well known to those of in the art, such as that depicted in FIG. 1, comprises two distinct phases: a stance phase 104 and a swing phase 106. The stance phase 104 for a given individual 108 begins with a “heel strike” 110 when the heel of the individual comes into contact with the ground, which is the last event indicating completion of the prior cycle for a given limb. Subsequent to the heel strike 110 the foot of the individual comes into complete contact with the ground across the entirety of the sole of the foot 112, before the heel begins to raise until “toe off”, which is where the toe of the foot for the given limb ends communication with the ground 116 and the stance phase 104 concludes. The swing phase 106 comprises the motion from toe off 118 through the heel strike 120 of the individual swinging the given limb forward to complete both the swing phase 106 and current gait cycle 102 comprising a walking event.

In accordance with currently accepted standards of care, the ability to understand issues affecting the gait cycles (e.g., walking pattern) of an individual and thereby provide an optimized and personalized care routine with respect to mobility related disorders is based on a number of qualitative and quantitative data points including, but not limited to, clinical interviews performed during short in-person meetings that typically take place, at best, every 3 or 6 months. Such clinical interviews may be supplemented by personal diaries and questionnaires for deferred review by the clinician, but which are not linked in time to symptoms the individual is reporting and are often affected by recall bias.

Disorders like Parkinson's present complex disease symptoms that vary as a function of time and with a given medication cycle. Consequently, the condition of an individual during a given visit with a clinician may not accurately reflect the degree and nature of his or her disability, limiting the clinician's ability both to capture an accurate image of the individual health and, consequently, maximize management of the disease afflicting the individual. According to the published evidence, level of capacity for an individual differs substantially when comparing in-clinic, supervised assessments with real-life assessments taking place in real-world, unsupervised situations. Moreover, in-clinic assessments do not capture that which an individual can actually do in his or her usual environment (i.e., level of performance for an individual).

To overcome these limitations in care, several studies have been conducted to explore the role of technology based objective measures, such as mobile and wearable technologies, as well as remote monitoring technologies. These types of solutions differ from the current standard of care by: (1) capturing, with greater frequency, the full complexity and diversity of symptoms that an individual is experiencing in his or her usual environment; (2) providing a more realistic and naturalistic portrayal of the mobility function for an individual; and (3) enabling for more closely monitoring of response of an individual to a given therapy.

Full body mobility analysis systems are currently required to obtain a full and objective assessment of movement as a basis for individually tailored clinical decision making and prognostication. In this context, optoelectronic tracking systems have been established as an accepted standard with respect to quantitative movement analysis. Such equipment, however, is expensive, restricted to controlled and calibrated environments, and requires a complex setup phase, all under the supervision of highly trained personnel. While wearable inertial sensors (e.g., inertial measurement units or “IMUs”) have been proven to be accurate, useful, and feasible for mobility analysis in clinical practice due to their relative low cost, light weight, and ease of use, such solutions are still highly tailored and do not provide ease of use to the individual. Moreover, such solutions require an in-person consultation with the individual. In between such assessments, motor symptoms and their fluctuations are still reported using diaries, questionnaires, and other subjective data collection modalities. The risk of poor adherence and limited time resolution, as well as the subjective nature of such information, raises concerns regarding the accuracy and reliability of data collected as such.

Accordingly, it is necessary to expand beyond conventional clinical assessments and aim for movement analysis in unsupervised, ecologically valid and relevant environments, which will allow clinicians to achieve a more accurate characterization of mobility issues that the individual is experiencing. Furthermore, there is a need for new methodologies of quantitative motion analysis and communication with an individual that provide improvements in terms of cost, time consumption, and influence of supervision.

Mobile health technologies, which can collect and connect clinical and non-clinical information as inputs to existing health informatics systems (e.g., electronic medical records), present a valuable solution to address aforementioned challenges. The key feature of mobile phones (i.e., pervasiveness, portability, ubiquity, and immediacy), make them a very attractive tool not only for improving, in a cost-effective way, continuous monitoring, clinical decision-making, and communication between stakeholders, but also to potentiate empowerment of an individual to self-manage his or her disease. Using mobile technologies for quantitative unsupervised movement analysis allows for the capture of motor symptom fluctuations and rare events associated with any given movement disorder while minimizing the effects of supervision.

### SUMMARY OF THE INVENTION

Embodiments of the present invention are directed towards systems and methods for the unsupervised collection mobility data and quantitative analysis with respect to the same. In accordance with one or more embodiments of the invention, a system for the unsupervised collection mobility data comprises a method for unsupervised monitoring of a mobility disorder experienced by a subject. The method in accordance with the present embodiment comprises collecting raw inertial data that represents local three dimensional (“3D”) orientation of the subject, extracting necessary inertial data from the raw inertial data, and analyzing the resultant extracted inertial data with respect to gait patterns to arrive at a gait classification on the basis of the analyzed, extracted data. The gait classification is provided as input to a recommendation process.

According to one or more embodiments, extracting necessary inertial data comprises conducting an attitude fusion to reorient and normalize the raw inertial data received from one or more sensors on a mobile device of the subject. Conducting an attitude fusion may comprise combining attitude estimates by integration of gyroscope measurements and direction obtained by accelerometer measurements to compensate for long term gyroscope integration drift and obtaining a global orientation of the mobile device. Alternatively, conducting an attitude fusion may comprise applying a rotation matrix to calculate a vertical component of an acceleration of the mobile device.

Analyzing in accordance with various embodiments comprises providing the extracted necessary inertial data as input to a gait classifier. Calculations provided by the gait classifier consists of calculations that include stride duration as the time difference between two ipsilateral heel-strikes; stance phase duration as the time between a heel-strike event and the following ipsilateral toe-off event; swing phase duration as the time between a toe-off event and the following ipsilateral heel-strike event; cadence as the number of strides per minute; stride length given by its linear relation with stride frequency, and acceleration variance; and stride speed as stride length divided by stride time. Finally, the method disclosed herein for unsupervised monitoring of a mobility disorder may comprise removing anomalous data from consideration.

### BRIEF DESCRIPTION OF THE DRAWINGS

The invention is illustrated in the figures of the accompanying drawings which are meant to be exemplary and not limiting, in which like references are intended to refer to like or corresponding parts, and in which:

FIG. 1 presents an illustration of a person competing a typical gait cycle while walking as is known in the prior art;

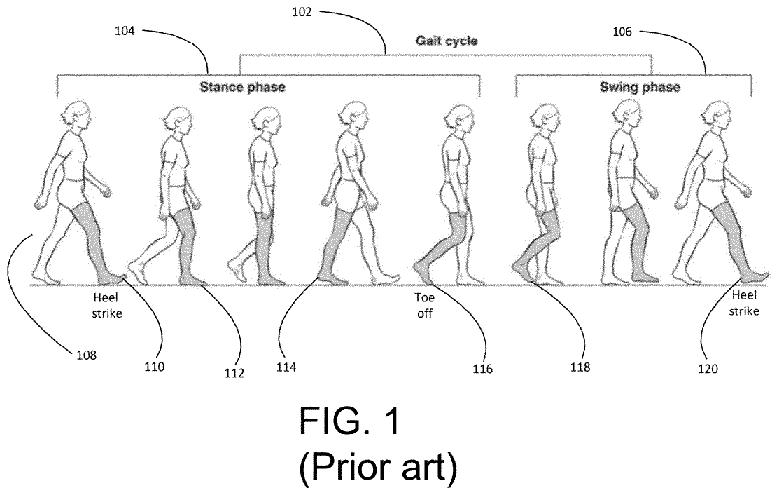

FIG. 2 presents a line drawing that illustrates hardware and software components for unsupervised monitoring of a movement disorder according to one or more embodiments of the present invention;

FIG. 3 presents a flow drawing that illustrates a process for unsupervised monitoring of a movement disorder according to one or more embodiments of the present invention;

FIG. 4 presents a flow drawing that illustrates a process for unsupervised monitoring of a movement disorder according to another embodiment of the present invention;

FIG. 5 presents a flow drawing that illustrates a process for gait detection and classification according to one or more embodiments of the present invention;

FIG. 6 presents a flow drawing that illustrates a process for mobile-based continuous risk of fall assessment according to one or more embodiments of the present invention;

FIG. 7 presents a flow drawing that illustrates a process for mobile-based continuous bradykinesia severity assessment according to one or more embodiments of the present invention;

FIG. 8 presents a flow drawing that illustrates a process for mobile-based continuous assessment of motor fluctuations according to one or more embodiments of the present invention;

FIG. 9 presents a flow drawing that illustrates a process for outlier detection according to one or more embodiments of the present invention;

FIG. 10 presents a graph illustrating heel strikes identified as peaks in a first derivative of the acceleration signal and toe offs as peaks directly in the acceleration in accordance with one or more embodiments of the present invention;

FIG. 11 presents a graph illustrating an exemplary anomalous event for removal in accordance with one or more embodiments of the present invention;

FIG. 12 presents a graph illustrating hidden toe-offs/heel strike pairs in which stride symmetry is assumed and a heel strike and a toe-off artificially placed in the middle of previous and next events in accordance with one or more embodiments of the present invention;

FIG. 13 presents a graph illustrating walking probability as a function, as well as vertical bars that correspond to times as which the patient was administered medication, in accordance with one or more embodiments of the present invention;

FIG. 14 presents a graph illustrating a medication duration window in accordance with one or more embodiments of the present invention;

FIG. 15 presents a graph illustrating deltas from t₀, tₘₐₓ, and t_{end}. t₀, tₘₐₓ, and t_{end} in accordance with one or more embodiments of the present invention;

FIG. 16 illustrates example line graphs representing extracted features from the raw inertial data in connection with FoG assessment;

FIG. 17 illustrates four example graphs, each representing a segmented signal in connection with respective ones of four extracted features from the raw inertial data shown and described with reference to FIG. 16;

FIG. 18 illustrates histograms corresponding to respective signatures of a respective segment in connection with four extracted features;

FIG. 19 illustrates an example report that identifies FoG and nonFoG percentages for a respective cluster;

FIG. 20 illustrates an example graph, which shows a distribution of FoG/nonFoG clusters in an example subject;

FIG. 21A presents a flow drawing that illustrates steps in an example process for mobile-based continuous assessment of participation, according to one or more embodiments of the present disclosure.

FIG. 21B presents a flow drawing that illustrates steps in an example process for mobile-based continuous assessment of FoG, according to one or more embodiments of the present disclosure;

FIG. 22 is an example report representing a tremor oscillator, which illustrates a graph representing calculated Tremor scores over a course of time (e.g., days); and

FIG. 23 presents a flow drawing that illustrates steps in an example process for mobile-based continuous tremor assessment, according to one or more embodiments of the present invention.

### DETAILED DESCRIPTION OF EMBODIMENTS OF THE INVENTION

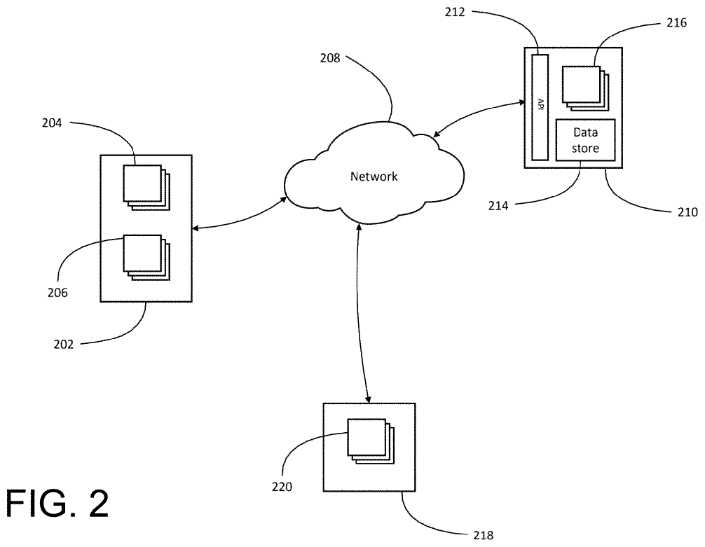

FIG. 2 presents a block diagram illustrating a system for the unsupervised collection of data regarding an individual, particularly with respect to mobility related disorders. In accordance with the embodiment of FIG. 2, a mobile device 202 of a user comprises a number of sensors 204 including, but not limited to, gyroscopes, magnetometers, accelerometers, etc., that provide data to other applications 206 running on the mobile device 202 regarding the position, attitude, movement, etc. of the mobile device 202 as a function of time.

Applications 206 running on the mobile device 202 include, but are not limited to, operating system software or other resident application to handle low-level hardware interaction and mediation of data rendered on its integrated display device, as well as one or more local program code components that perform unsupervised collection of mobility data. The local program code components 206 may collect data from the one or more sensors 204, with such local program code components 206 performing one or more steps comprising a movement analysis, which is described in greater detail herein.

The system 200 provides a platform that pairs such local program code components 206 running on a mobile device 202 to continuously monitor a given individual in unsupervised settings. The local program code 206 executing at the mobile device 202 opens communication channels with one or more sensors on the device 206 including, but not limited to, one or more gyroscopes, accelerometers, and magnetometers. Data that the local program code components 206 monitor and/or collect may be sent from the mobile device 202 over a network 208 for storage 214 by cloud-based services 210, which may comprise further cloud-based processing of the monitored data by cloud-based program code components 216, as well as presentation of data through accompanying applications that dynamically display both collected and processed information, as well as allows clinicians to remotely interact with mobile devices of an individual 202 through the use of a clinician's device 218.

The local program code components 206 running on the mobile device 202 allows for passive, long-term, unsupervised functional mobility quantification and position tracking of the individual in any environment. The local program code components 206 also provide for remote active capacity testing (e.g., one-minute balance test, finger tapping exercises, a walk test, etc.) and responding to on-demand (e.g., one time or periodic) self-reported questionnaires. The local program code components 206 allow for the digital collection of these various data points, which aids in downstream storage and processes, as well as eliminates the possibility of transcription errors, e.g., when digitizing personal diaries and questionnaires as is known in the prior art. As indicated above, specific collection and processing methodologies are described in greater detail herein.

The combination of one or more of these data points allows a clinician to easily quantify and track progress and treatment response over time. The local program code components 206 may make use of a communication channel over the network 208 that the mobile device 202 provides to allow the individual to communicate with his or her clinician using a clinician device 218, which may further comprise allowing the user to communicate with his or her clinician in the management of his or her disease, e.g., medication updates such as changes to dosage, frequency, etc., reporting rare events, symptom changes, etc.

According to the embodiment of FIG. 2, the local program code components 206 may further make use of a communication channel over the network 208 that the mobile device 202 provides to invoke functionality of cloud-based program code components 110 by way of the application programming interface (“API”) 212. Cloud-based program code components 216 comprise access to a data storage facility 214 to which the local program code components 206 may push the aforementioned collected data.

In addition to the foregoing, clinician and a given individual use a cloud-based API to access communication functionality provided thereby. Clinician may utilize his or her clinician device 218, which may be any manner of digital computing device, such as a general-purpose PC, a smartphone, tablet, etc., to access communications functionality that the cloud-based program code 216 provides by way of the API 212. As such, the clinician may initiate or respond to communications that a given individual may send or receive with his or her mobile device 202. Additionally, the cloud-based program code components 216 may allow the clinician to use software 220 executing on his or her clinician device 218 to access mobility data. The cloud-based program code components 216 may transmit the data for presentation on the clinician device 218 as one or more web-based dashboards, e.g., one or more static or dynamic web pages that software at the clinician device 218 renders for presentation on an associated display device.

According to one or more embodiments, an individual equipped with a generic mobile device 202 that provides for storage of digital data from one or more sensors 204 initiate processing by one or more local program code components 206 for the unsupervised collection of data. As the local program code components 206 continue executing on the mobile device, the program code components 206 collect mobility data of the individual in real-world, unsupervised conditions. The data that the local program code components 206 collect may be stored locally or at the data store that is part of the cloud-based services 210. According to various embodiments, processing of such data that the local program code components 206 collect may be by the local program code components 206, cloud-based program code components 216, or combinations thereof. Advantageously, the collection of such mobility data over time allows for the processing of such data, e.g., through the use of machine learning techniques, to make assessments with respect to future mobility outcomes, such as risk of fall assessments, bradykinesia severity, motor fluctuations, etc.

The cloud-based services 210 provide for a robust reporting facility that the API 212 exposes. Using such reporting facility, for example, the local program code components 206 may issue an API call for a weekly report on the functional mobility of the individual, as well as his or her capacity in one or more active tests. In response, the cloud-based program code components 216 may collected such processed data for transmission back to the requesting device. Alternatively, the cloud-based program code components may process raw data in response to the request for transmission back to the requesting device. Similarly, program code 220 at the clinician device 218, as is explained in greater detail herein, may issue an HTTP request or API call for various types of reports on an individual under his or her care, e.g., a functional mobility report for a given individual, capacity of a given individual on one or more active tests, etc. In response, the cloud-based program code components 216 may collected such processed data for transmission back to the clinician device 218. Alternatively, the cloud-based program code components may process raw data in response to the request for transmission back to the clinician device 218.

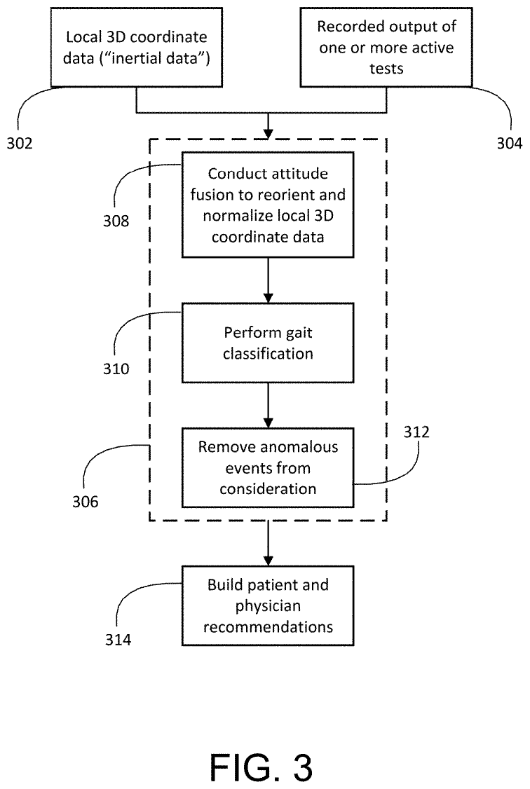

FIG. 3 presents a flow drawing that illustrates a process for unsupervised monitoring of a movement disorder according to one or more embodiments of the present invention. According to the exemplary program flow of FIG. 3, the process begins with the collection of various types and amounts of raw data. One such data type is local 3D orientation data, also referred to herein as “inertial data” or “movement data”, step 302, which may be continuously or periodically received from one or more sensors on the mobile device, e.g., the combination of an accelerometer, a gyroscope and magnetometer, solely the output of either sensor, other positional sensors alone or in combination, as well as in combination with the foregoing, etc. Other input raw data may include the recorded output of one or more active capacity tests, step 304, such as a finger tapping test that actively measure the impairment in voluntary movement (e.g., bradykinesia) of the individual and provide an additional set of mobility data points in addition to the inertial data that is continuously or periodically received from sensors on the mobile device.

The raw inertial data and data from one or more active test are provided as inputs to a sub-process 306 that extracts necessary inertial data, analyzes the resultant data with respect to gait issues or patterns, and performs classification as input to a recommendation process. According to various embodiments, the data pipeline is prepared to collect inertial data from any sensors that are present on the mobile device of the user including, but not limited to, accelerometer, gyroscope and magnetometer.

After ingestion of the inertial data, the device position independent gait-related feature extraction process conducts attitude fusion to reorient and normalize the local 3D orientation data received from the one or more sensors on the mobile device, step 308. According to one or more embodiments, sensor synchronization is achieved through linear interpolation and spherical linear interpolation (for orientation data), with sensor fusion performed using a Madgwick's gradient descent IMU orientation estimation. Application of this technique combines attitude estimates by integration of gyroscope measurements and direction obtained by accelerometer measurements, thereby compensating for long term gyroscope integration drift, to obtain the global orientation of a mobile device.

Alternatively, program code components may apply a rotation matrix to calculate the vertical component of the acceleration of the mobile device whereby all downstream analysis depends on the acceleration signal. Before isolation of the acceleration signal, however, it is necessary to transform the mobile device local orientation data (x, y and z) of the to a normalized, global orientation system that is useful for subsequent analysis of such data, which, in this case, is the vertical component of the acceleration in relation to the world. To obtain this data point, one or more embodiments interpolates the signals to common timestamps through linear interpolation in intervals of 1000/freq (wherein “freq” is equal to the average collection frequency). To rotate accelerometer data and ultimately recover the global vertical acceleration component, one of several algorithms may be utilized, depending on the available sensors.

First, consider those mobile devices where only an accelerometer is present. In such instances the algorithm begins by letting the vector

a=(aₓ,a_{y},a_{z})

represent the three acceleration components of the sensor at a given point. For a chosen interval, the technique estimates the gravity component of a on each axis by averaging all its readings:

v=(vₓ,v_{y},v_{z})

Vector m, representing the component of a caused by the user's motion is given by:

m=(aₓ−vₓ,a_{y}−v_{y},a_{z}−v_{z})

The global vertical direction can be obtained by:

v/∥v∥

Finally, the global vertical component of vector m is given by:

m·(v/∥v∥)

Next, consider those mobile devices where the magnetometer and accelerometer are present, but not the gyroscope. In such instances, the quaternion parameterizing the orientation is derived from a least-squares optimization. Consider the vectors

a=(aₓ,a_{y},a_{z})

and

m=(mₓ,m_{y},m_{z})

to represent the normalized accelerometer and magnetometer observations. Further, consider the vectors

a=(0,0,1)

and

mᵣ=(m_{N},0,m_{D})

to represent the normalized reference vectors in the global reference frame r (North-East-West).

Continuing with the foregoing, where:

m_{D}=aₓmₓ+a_{y}m_{y}+a_{z}m_{z}

and

m_{N}=\sqrt{(1−m_{D}²)}

the unitary quaternion solution is given by

q/∥q∥

where

q=(q₀,q₁,q₂,q₃)

with

q₀=(a_{z}−1)(m_{N}+mₓ)+aₓ(m_{D}−m_{z})

q₁=(a_{z}−1)m_{y}+a_{y}(m_{D}−m_{z})

q₂=a_{z}m_{D}=a_{z}m_{N}−m_{z}

q₃=−a_{y}(m_{N}+mₓ)+aₓm_{y}

This quaternion can be converted to its rotation matrix representation. Acceleration in the global reference frame is given by the dot product between this matrix and the original acceleration vector (in the sensor frame).

Beyond the foregoing, if the magnetometer, accelerometer, and gyroscope are present, a Madgwick filter may be used. Again, let the vector

a=(aₓ,a_{y},a_{z})

represent the normalized accelerometer measurements in the sensor frame. The initial guess is given by the SAAM algorithm, obtaining quaternion q. A gradient descent step (orientation increment from accelerometer measurements) is performed where ƒ represents the objective function:

$$f = [ \begin{matrix}2 ( {q}_{1} {q}_{3} {q}_{0} {q}_{2} ) - {a}_{x} \\ 2 ( {q}_{0} {q}_{1} + {q}_{2} {q}_{3} ) - {a}_{y} \\ 2 ( 0.5 - {q}_{1}^{2} - {q}_{2}^{2} ) - {a}_{z}\end{matrix} ]$$

and j represents the objective function's jacobian:

$$j = [ \begin{matrix}- 2 {q}_{2} & 2 {q}_{3} & - 2 {q}_{0} & 2 {q}_{1} \\ 2 {q}_{1} & 2 {q}_{0} & 2 {q}_{3} & 2 {q}_{2} \\ 0 & - 4 {q}_{1} & - 4 {q}_{2} & 0\end{matrix} ]$$

The normalized gradient is given by

g=j^{T}·ƒ

and the attitude update component from the accelerometer measurements is given by

−β(g/∥g∥)

Regarding the gyroscope, the orientation increment from measurements received off of the gyroscope is given by numeric integration as follows:

0.5⊗(^{s}w)

where

w=(wₓ,w_{y},w_{z})

represents the gyroscope measurements in the mobile device frame and

^{s}w=[0,wₓ,w_{y},w_{z}]

The quaternion q is updated by adding

[−β(g/∥q∥)+0.5q⊗(^{s}w)]*Δt_{(data fusion)}

where t is the sampling period and β represents the magnitude of the gyroscope measurement error. q can then be normalized and used as the initial guess for the following frame. Accordingly, regardless of the sensors available and the corresponding technique that the algorithm utilizes to reorient and normalize the orientation data, the vertical component of the acceleration is obtained for use in gait classification.

The algorithm according to the present embodiment uses the reoriented and normalized orientation data as input to a gait classifier, step 310. Several exemplary parameters may be calculated as part of gait classification: stride duration as the time difference between two ipsilateral heel-strikes; stance phase duration as the time between a heel-strike event and the following ipsilateral toe-off event; swing phase duration as the time between a toe-off event and the following ipsilateral heel-strike event; cadence as the number of strides per minute; stride length given by its linear relation with stride frequency, and acceleration variance; stride speed as stride length divided by stride time.

The obtained vertical component of the acceleration signal may be split into windows of ˜5.33 seconds (320 points at 60 Hz) and, for each segment, two features calculated: power spectra in the frequency bands 0.1-3 Hz and 0.1-10 Hz. These two features may be fed to a supervised C-Support Vector Classification with a regularization parameter C of 0.1 and a kernel coefficient gamma of 0.01. Adjacent segments that are classified as gait may then be combined to obtain a continuous acceleration signal that corresponds to a gait period, which may be made available to other components and processes comprising the various embodiments of the invention.

According to one or more embodiments, gait events are detected using a peak detection algorithm on the global vertical acceleration time series, which may be filtered, e.g., with a 4^{th} order Butterworth bandpass filter with critical frequencies at 0.1 and 2 Hz, as well as on its respective derivatives. Peaks on the acceleration may correspond to toe-off events, whereas peaks on its derivatives correspond to heel strikes.

As indicated above, the noisy acceleration signal may be smoothed using a bandpass filter, such as a Butterworth filter, with two critical frequencies of 0.1 and 2.0 Hz and an order of 4. This filtering step discards high frequency events such as tremor, which may be, e.g., physiological or PD-associated, signal noise, etc. Such filtering also contributes to attenuation of the differences between several smartphone locations on the body of the individual; with the selected critical frequencies, only gait-associated events are captured.

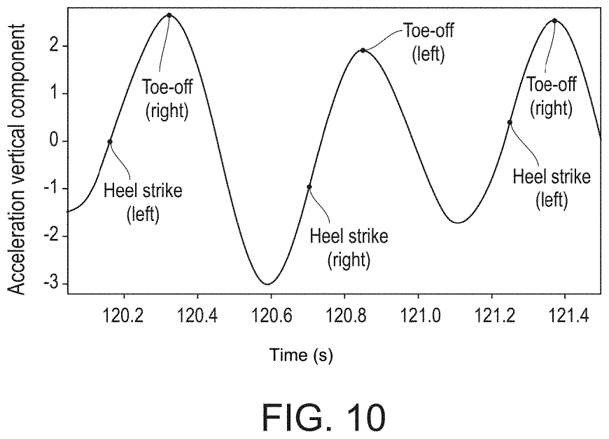

From the filtered acceleration signal, key gait events are identified: heel strikes and toe offs. In accordance with certain embodiments, heel strikes are identified as peaks in the first derivative of the acceleration signal, whereas toe offs correspond to the peaks directly in the acceleration, which is illustrated in FIG. 10.

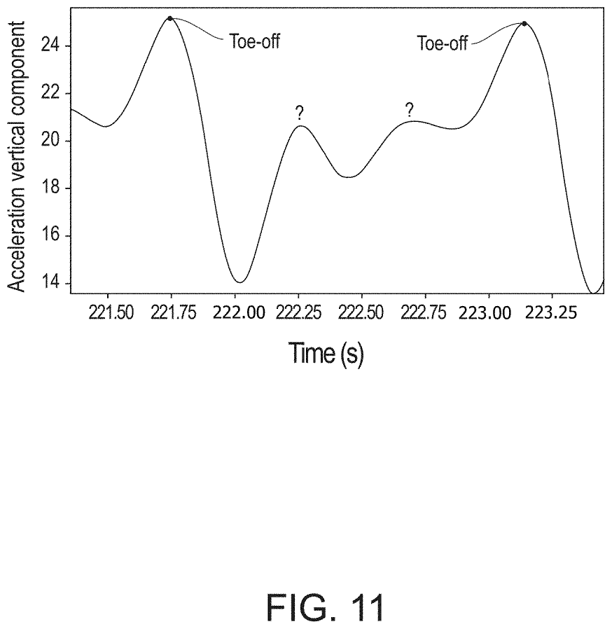

In certain embodiments, consideration for analysis is limited to those events received from the local program code components on the mobile device with corresponding events on the criterion, thereby removing anomalous events from consideration, step 312. An exemplary anomalous event for removal is represented in FIG. 11.

Sometimes it happens that there is the observation of two peaks (toe-offs) that are significantly lower than the previous and the next one. In these cases, the specific mobility event transpiring is unclear since the two higher peaks should correspond to toe-offs of the same side. Hence, embodiments of the present algorithm are configured to ignore these strides. According to certain embodiments, a stride is ignored when there are two peaks that are 30% smaller than the previous and the next ones.

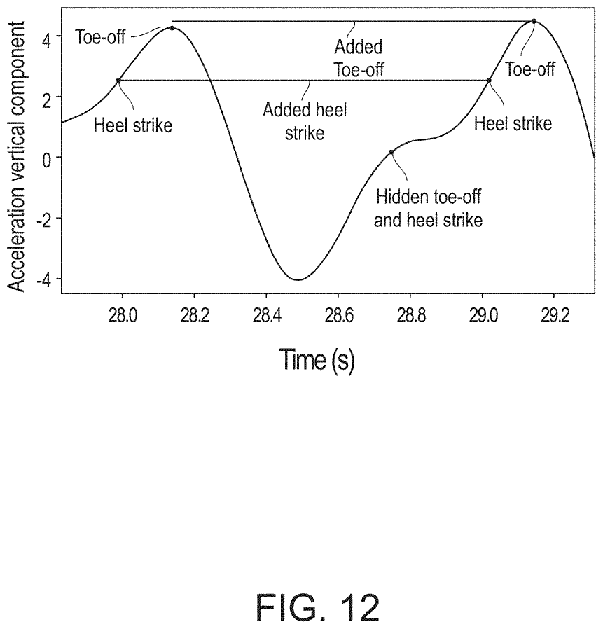

In addition to the foregoing, within the process of filtering and event detection there are sometimes hidden toe-offs/heel strike pairs. In such instances, embodiments assume symmetry for stride and artificially place a heel strike and a toe-off exactly in the middle of previous and next events, illustrated in FIG. 12. After this protocol, a vector of heel strikes and toe-offs is obtained, which may be ready for feature calculation.

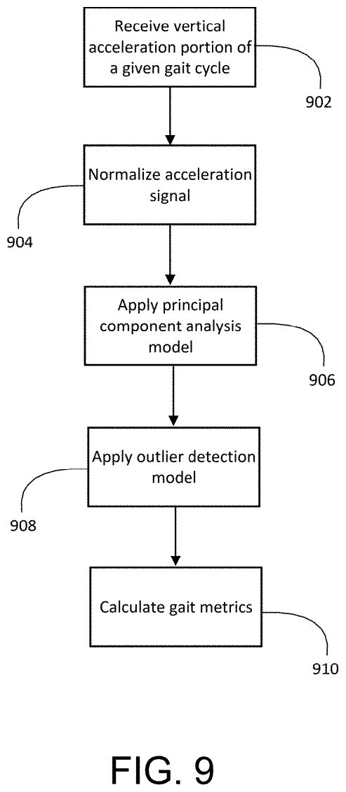

In the addition to the foregoing, embodiments of the present methodologies may identify and remove anomalous events through application of an outlier detection method, one or more embodiments of which is illustrated in FIG. 9. The outlier detection method of FIG. 9 may begin with the collection of input data. For example, the process may use the vertical component of the acceleration signal, step 902. Extraction of the vertical component of the acceleration signal may be performed for a given stride after the gait classification and removal of anomalous events.

With the selected signal components received from a given gait cycle, such acceleration signal may be normalized, step 904. The number of data points of the acceleration signal of a stride is variable, e.g., depending on the stride time and on the sensor sampling frequency. To obtain a signal that is ready for or otherwise compatible with downstream model processing, the acceleration signal may be linearly interpolated into a multipoint vector, e.g., a vector of one hundred data points.

With the normalized data, step 904, the process applies a principal component analysis model, step 906. According to one or more embodiments, a principal component analysis (“PCA”) model is applied to the data points comprising the vector, whereby a number of the principal components are retrieved, e.g., the first four. Similarly, an isolation forest model may be applied to the retrieved components to determine if the stride represents an outlier, step 908. According to various embodiments, the PCA model and the isolation forest model may be pre-trained. For example, both the PCA and the isolation forest models may be trained using ˜56k data points corresponding to strides of individuals with PD whereby smartphones are placed in different body locations.

Gait features may be extracted from the filtered event stream, step 910, as well as with, without, or replaced by derivatives or additions thereof. Some exemplary features include, but are not limited to:
stride duration::temporal distance between heel strike n and n+2
stance duration::temporal distance between the heel strike and the toe-off of the same side
swing duration::temporal distance between the toe-off and the heel strike of the same side(stride=stance+swing)
cadence::60/stride duration (strides per minute)

According to certain embodiments, the interquartile range rule may then be applied to a given gait metric to select values that are larger (lower) than 1.5×IQR+1^{st} (−3^{rd}) quartile, where IQR represents the interquartile range.

Turning back to FIG. 3, with the conclusion of step 312 sub-process 306 makes available data for building recommendations, step 314. As is described in greater detail herein, exemplary recommendations are provided with respect to assessments such as risk of fall, bradykinesia severity, motor fluctuations, etc. It should be noted that processing within sub-process 306 returns to step 308 such that there is continuous collection and processing of mobility data in real-world, unsupervised settings for a given individual. As the data processing of sub-process 306 produces additional data, such data is made available data for refining or building patient and clinician recommendations, step 314.

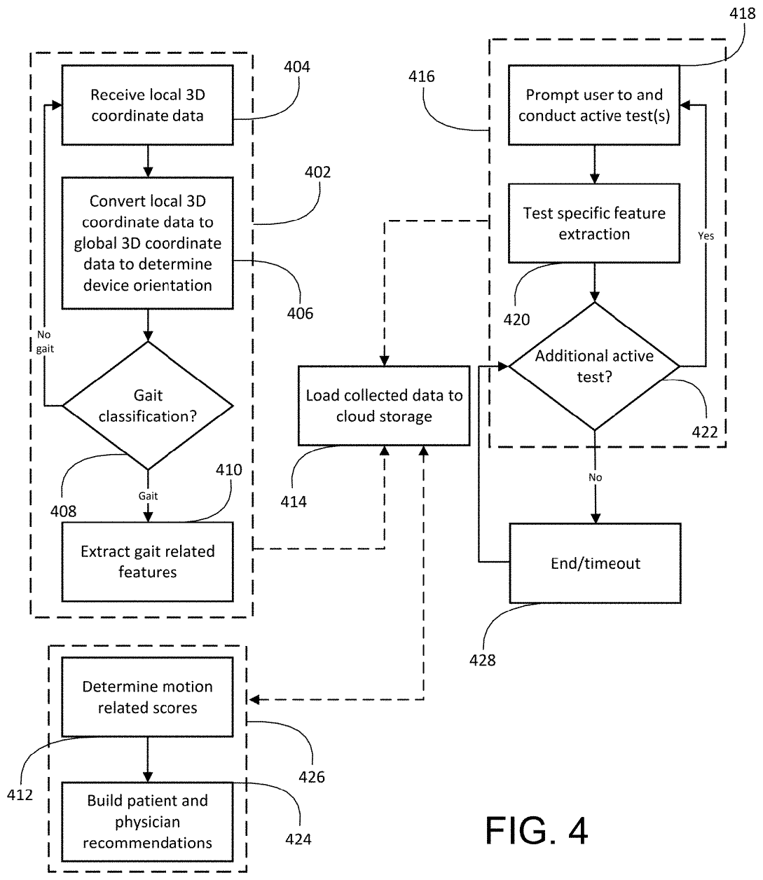

FIG. 4 presents a flow drawing that illustrates a process for unsupervised monitoring of a movement disorder according to another embodiment of the present invention. In accordance with the alternative embodiment of FIG. 4, the algorithm implements two subprocesses: a first sub-process to collect inertial data for gait classification and feature extraction, step 402, and a second sub-process for collection and processing of active tests, step 416.

The first sub-process for the collection of inertial data for gait classification and feature extraction, step 402, starts with the receipt of local 3D orientation data. In accordance with various embodiments of the invention, local program code components may receive local 3D orientation data from one or more sensors on a mobile device, step 404, including, but not limited to, gyroscopes, magnetometers, and accelerometers. Program code, which may be resident locally at the mobile device or hosted remotely on one or more cloud-based services, receives this raw inertial data for further processing in accordance with the embodiments described herein.

Program code converts that local 3D orientation data of the mobile device to a normalized, global orientation system that is useful for subsequent analysis of such data step 406, which, in this case, is the vertical component of the acceleration in relation to the world. Depending on the combinations of sensors that are available on the mobile device, the method may make use of one of several disparate techniques for isolation of the vertical component of the acceleration from the received raw data.

A combination of local and/or cloud program components receive the normalized, global orientation data, or selected components thereof, for processing to determine whether or not the data represents gait, step 408, as opposed to some other motion of the patient. A number of disparate techniques can be used to determine the presence of gait on the basis of the incoming data. For example, a model may be trained on the basis of a substantially large set of training data that represents gait, whereby the trained model is used by a machine learning process to determine if an unlabeled data item under consideration is indicative of gait. Where the incoming data is not indicative of gait, step 408, program flow returns to step 404 with the receipt of additional raw inertial data for processing and analysis.

Where the check at step 408 indicates that the data represents the presence of gait, processing continues with the extraction of gait related features, step 410. In accordance with one or more embodiments, the local and/or cloud-based program code components are operative to process data indicative of gait to extract features relating to stride, stance, and swing duration, cadence, stride length, energy, etc. It should be apparent to those of skill in the art that the incoming data lends itself to the extraction of additional features regarding gait, all of which fall within the scope of embodiments of the present invention.

Extraction of features proceeds with loading such gait related features, which may comprise raw and/or processed inertial data, as well as any additional gait related data, for storage in a cloud based repository, step 414. According to one or more embodiments, the data store is organized on a per-patient basis. The sub-process 402 continues to with the receipt of additional local 3D orientation data, step 404 and corresponding processing thereof.

The second sub-process for the collection of active tests data, step 416, starts with program code components, which again may comprise various combinations of local program code components and cloud-based program code components, prompting the patient, e.g., through display prompts on the mobile device, to initiate a given active test, step 418. The user engages in the given active test with the results of the test recorded, at least temporarily, on local storage at the mobile device if not loaded into cloud storage, step 414, as an intermediate out-of-process step.

Test specific features are extracted from the resultant data collected via the mobile device in response to administration of the given active test, step 420. Such test specific features extracted from the resultant data are loaded into the data store provided by the cloud-based services, step 414. Loading data to the data store, step 414, may be performed by the mobile device through the cloud-based services where the mobile device conducts such test specific feature extraction, although the cloud services may push any data at the cloud-based program code components to the data store.

Program code, which may be running on local or remote processors, performs a check to determine if any additional active tests are available for execution by the patient, step 422. Where the check determines that additional active tests require execution, program flow returns to step 418 with the administration of the subsequent active test, as well as associated data processing and collection. Otherwise, where no additional active test is available, step 422, sub-process 416 terminates at end step 428. In certain embodiments, the end step 428 comprises a timeout, which may be a clinician or systemwide setting, the expiration of which causes activation of the second sub-process 416 and activation of a check to determine any active tests are available for execution by the patient, step 422.

As data accumulates in the data store, step 414, a third sub-process 426 operates to provide determinations on the basis of the extracted gait related features and results of one or more active tests conducted by the patient. Program code accesses the data store hosted by the cloud-based services so as to determine one or more motion related scores, step 412. For example, analysis that the program code performs when executed by a processor, e.g., at the cloud-based services, may provide an assessment as to a fall risk that a given patient poses. Similarly, analysis may provide an assessment regarding bradykinesia severity or motor. The sub-process 426 may push the resultant data back into the data store for persistent storage, step 414.

Clinician recommendations are built on the basis of the one or more motion related scores, which the system provide to the clinician in the form of one or more reports, step 424. The one or more reports may be static or dynamic web pages that are built by cloud-based program code components, which may be built in response to an HTTP request to the cloud-based services receives. Where the reports are web pages, they are transmitted over a network to a clinician device executing a web browser that is operative to receive, render, and display the data in the web page(s). It should be noted that as the data store continues to receive additional data for storage, such data is available to the third sub-process 426 so as to continually generate new and refine existing motion related score on the basis of new or additional data, step 412, as well as update reports and recommendations that are based thereon.

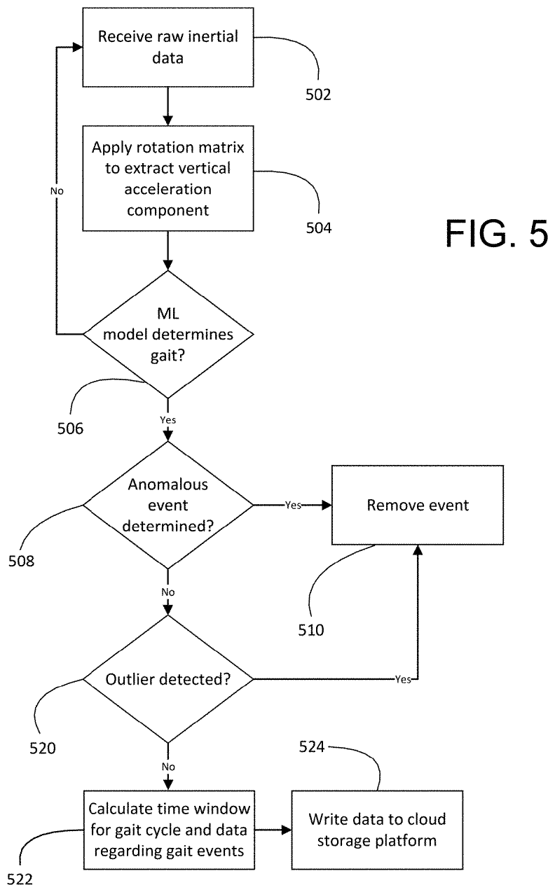

Providing additional clarity into the gait classification process, FIG. 5 presents a flow drawing that illustrates a process for gait detection and classification according to one or more embodiments of the present invention. As with the embodiments of FIGS. 3 and 4, the embodiment of FIG. 5 also begins with the receipt of raw inertial data from one or more sensors on the mobile device of the patient, step 502. Although other techniques are available, step 504 continues with the application of a rotational matrix to extract the vertical acceleration component from the raw inertial data, step 504.

The vertical acceleration component that the program code extracts from the raw inertial data is fed to a machine learning process with access to a model trained to recognize gait and the various events comprising the gait cycle. The program code performs a check to determine if the machine learning process determines the presence of gait in the extracted vertical acceleration component, step 506. If the machine learning process determines that the extracted data does not represent gait, program flow returns with the continued receipt of raw inertial data, step 502.

Where the machine learning process determines that gait is, in fact, present in the extracted vertical acceleration component, step 506, the program code performs a check to determine if the gait cycle that the machine learning process is an anomalous event, step 508, which may comprise processing by another machine learning process specifically trained to identify and resolve anomalous events. Similarly, the program code performs a check to determine if the data represents an outlier of any kind, step 520. In the event that either contingency evaluates to true, step 508 or step 520, the event under consideration is discarded. According to some embodiments, the data store maintains such “discarded” data, which is only sequestered from the production data stream upon which the system calculates motion related scores, for further analysis and processing by a system administrator or clinician.

Where the machine learning process identifies a gait event that passes anomalous event and outlier detection scrubbing, program flow continues with the calculation of time windows for gait cycle analysis and other related data regarding the gait event, step 522, e.g., power, cadence, etc. The cloud-based services write the resultant data to a data store made available by the cloud-based services, step 524. As such, the gait features are available for further processing by downstream processes, such as the calculation of one or more motion related scores, either alone or in combination with features extracted from one or more active tests.

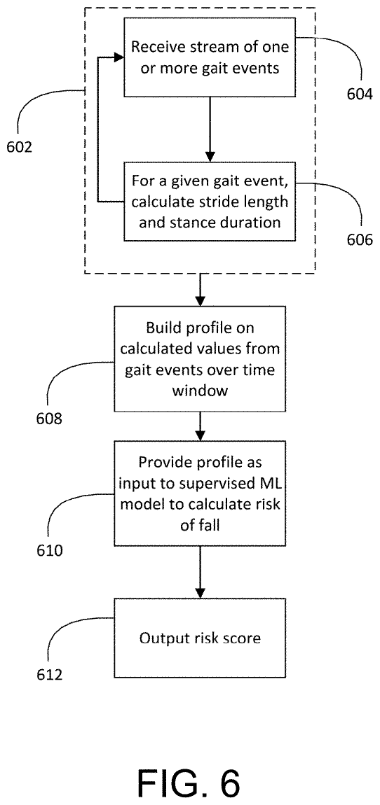

As described above, program code may conduct attitude fusion to reorient and normalize the local 3D orientation data received from the one or more sensors on the mobile device, e.g., raw inertial data, so as to extract device position independent gait-related features therefrom. These gait-related features, some of which are set forth above, serve as inputs to various assessments that the program code components may conduct to hereby provide the clinician with additional detail regarding the health of the patient, disease progress, and the manner in which the patient may be responding to any therapies that the clinician is prescribing. FIG. 6 presents one exemplary embodiment of a process for providing a risk of fall assessment if, which may be provided to the clinician and/or patient on a periodic or continuous basis.

In accordance with the embodiment of FIG. 6, a gait-feature sub-process 602 is operative to receive input data in the form of one or more gait events, step 604, which may comprise gait events as identified by embodiments of the gait classification pipeline described herein. For a given input gait event, step 604, program code is operative to calculate stride length and stance duration for the given input gait event, step 606. The sub-process 602 of receiving gait-related events for processing, step 604, to determine gait-related features that are specific to a risk of fall assessment, step 606, may comprise a continuous, near continuous, or periodic flow of ecologically assessed gait metrics. To evaluate the risk of fall, the process utilizes two features: stride length and stance duration. According to certain embodiments program code also calculates the average of both the stride length and stance duration features.

As the program code determines gait-related features from the incoming stream of gait events, the program code builds a profile for the patient over a time window, step 608. According to one or more embodiments, the risk of fall profile is built on selected average gait events collected during a previous two week period, which may comprise collecting such data for two weeks and calculating the average of all events.

Program code evaluates data in the risk of fall profile to provide the clinician with an assessment regarding risk of fall for the patient, step 610. According to one or more embodiments, a supervised machine learning model is used to estimate the Timed Up and Go (“TUG”) value, which provides an assessment with respect to mobility, balance, walking ability, and fall risk in older adults or individuals suffering from a variety of mobility disorders. The TUG test, in which a clinician times a patient standing from a seated position, walking a set distance, and then returning to the seated position, is considered the standard of care with regard to screening tools used to assist in identifying patients at risk of falling. The risk of fall is defined as low for TUG below 7.95 s, moderate between 7.95 s and 11.5 s and high above 11.5 s.

The program code may implement one or more various machine learning techniques in providing its risk of fall assessment for the patient. For example, an Epsilon-Support Vector Regression model may be trained through the use of a set of labeled training data, whereby one or more unlabeled data items are presented to predict clinically assessed TUG. A polynomial kernel of order 3 and a regularization parameter of 100 may be used, although the use of other tuning parameters falls within the scope of the various embodiments of the present invention.

With the trained model, TUG may be continuously evaluated, with the risk score also output or updated on a continuous or periodic basis, step 612. One outcome of the process comprises fall evaluation, which according to one or more embodiments is classified as low, moderate, or high, although other classification scales or metrics may be used. Program code may provide the fall evaluation value to the patient on his or her mobile device, as well as to the clinician on his or her clinician device. Program code at the cloud-based services may periodically generate reports that include the fall evaluation value as one among several data points in a given report.

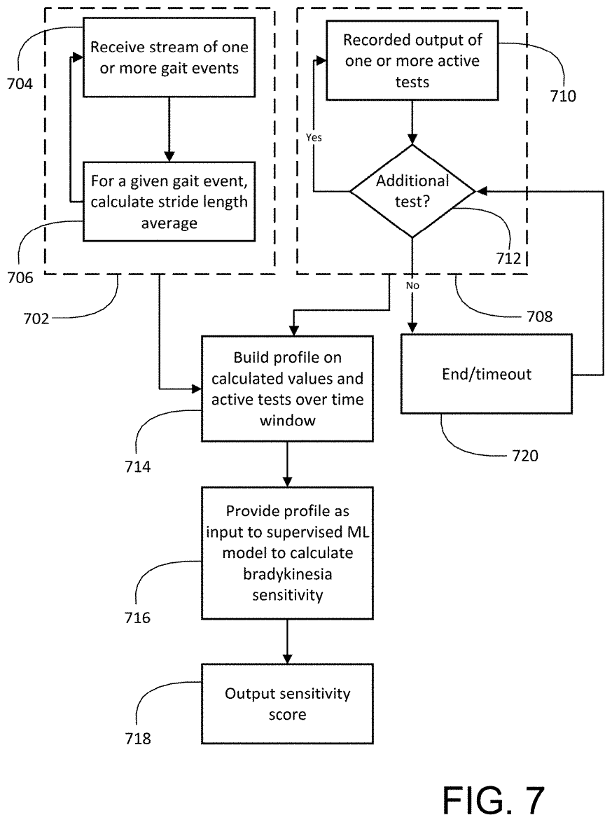

Another assessment that the program code components may conduct to provide the clinician with additional detail regarding the health of the patient, disease progress, and the manner in which the patient may be responding to any therapies that the clinician is prescribing is with respect to bradykinesia severity. FIG. 7 presents a flow drawing illustrating a process for mobile-based continuous bradykinesia severity assessment according to one or more embodiments of the present invention. Bradykinesia generally refers to slowness of movement and may manifest itself in various forms, such as a reduction in automatic movements, difficulty initiating movements (like getting up out of a chair), the appearance of abnormal stillness, etc. A bradykinesia assessment in accordance with the present embodiment comprises the combined analysis of both gait-related features and hand-related features. Accordingly, input to a bradykinesia assessment utilizes two disparate sources of data, which the process collects through the use of two distinct sub-processes described herein.

The first subprocess 702 comprises the continuous flow of ecologically assessed gait-related features, step 704, and, in particular, the stride length, step 706. According to one or more embodiments, previously described processes for the continuous flow, collection, and processing of gait-related features are hereby utilized to collect gait-related events and extract gait-related features therefrom. According to certain embodiments, both values for gait-related features, as well as averages therefore over a window of time, are written for persistent storage on a data storage device.

While the first sub-process, step 702 is collecting the continuous flow of ecologically assessed gait-related features, a second sub-process, step 708, which may be operating in parallel with the first subprocess, is directed towards the collection of data from one or more active tests. Accordingly, the second sub-process records the output of a given active test, step 710, which may comprise writing the results of the given active test to a persistent storage device for later access and use. For example, an active test may comprise an active finger tapping test that is performed every week and consists of a 1-minute test where the patient is tapping a circle in the smartphone screen as fast as he or she can. From this test, the program code components calculate the standard deviation of the time between sequential touches.

After collection of the output from the given active test, the program code performs a check to determine if there are additional active tests that require completion by the patient, step 712. Where there are additional active tests that require completion by the patient, program flow returns to step 710 with the patient being presented with the subsequent active test. Where there are no additional active tests, step 712, the sub-process may end, step 720. The end state, step 720, may be a timeout state such that after expiration of a set amount of time, program flow may return to step 712 and execute a check to determine if there are additional active tests that require completion by the patient.

As the program code determines gait-related features from the incoming stream of gait events, step 702, as well as relevant features from one or more active tests, step 708, the program code builds a profile for the patient over a time window, step 714. According to one or more embodiments, a bradykinesia profile is built on selected gait-related features and tapping-related features collected during a previous two week period, which may comprise collecting such data over a window of time and calculating the average of all events.

Program code evaluates data in bradykinesia profile to provide the clinician with an assessment of possible bradykinesia that the patient experiences, step 716. According to one or more embodiments, a supervised machine learning model is used calculate a bradykinesia severity score on the MDS-UPDRS scale, which provides an assessment with respect to the severity and progression of a mobility related disease, such as Parkinson's Disease. The MDS-UPDRS scale provides a gold standard against which clinicians may monitor the response to medications used to decrease the signs and symptoms of various movement disorders. The test is broken into multiple parts, with part three (3) directed towards motor examination.

The program code may implement one or more various machine learning techniques in providing its bradykinesia assessment for the patient. For example, a supervised machine learning model (regressor) may be trained through the use of a set of labeled training data, whereby one or more unlabeled data items are presented to predict an estimate of clinically assessed bradykinesia severity. According to one or more embodiments a linear equation is used such that the bradykinesia score is equal to −19.68*stride_length+105.79*std_time_between_touches+29.97. The output score represents an estimation of the of the bradykinesia parts of MDS-UPDRS scale, which may comprise the sum of scores related to sections 3.4, 3.5, 3.6, 3.7, 3.8, 3.14.

With the trained model, bradykinesia severity may be continuously evaluated, with the bradykinesia score also output or updated on a continuous or periodic basis, step 718. Program code may provide the bradykinesia score or value to the patient on his or her mobile device, as well as to the clinician on his or her clinician device. Program code at the cloud-based services may periodically generate reports that include the bradykinesia severity as one among several data points in a given report.

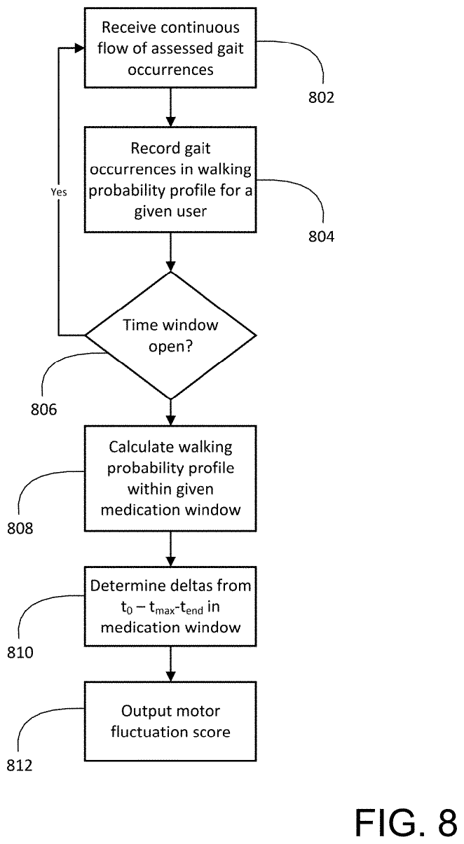

In addition to providing assessments regarding risk of fall and bradykinesia severity, the system may be further operative to provide assessments with respect to motor fluctuations. FIG. 8 presents a flow drawing illustrating a process for mobile-based continuous assessment of motor fluctuations according to one or more embodiments of the present invention.

As with other assessment methodologies described herein, input data to the motor fluctuations assessment comprises the receipt of a continuous flow of ecologically assessed gait occurrences, step 802. According to one or more embodiments, the assessment only concerns the temporal features of a given gait event, e.g., the beginning and the duration of the gait event. According to some embodiments, the process may utilize stride time or length, cadence, etc. as opposed to or in conjunction with gait metric features.

The process of FIG. 8 records the collected gait occurrences, step 804, which may comprise writing of such data to a persistent storage device, such as a network accessible data store. According to one or more embodiments, writing of such data comprises the creation of a walking profile over a window time, such as a month. Where the time window is not yet open, step 806, e.g., an insufficient amount of data has been collected, program flow returns to step 802 with the continued collection of gait occurrences. Where sufficient events that have been collected over the time window to create a daily profile that is patient specific, step 806, further processing occurs.

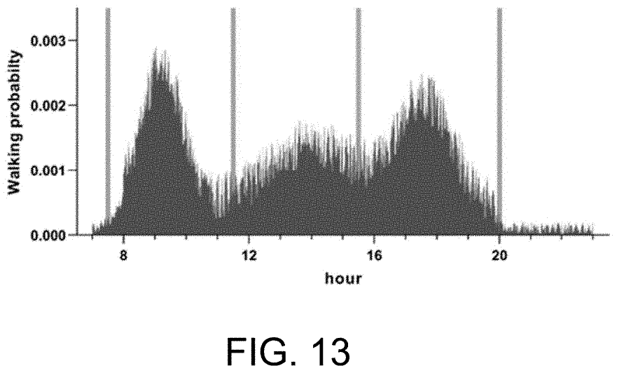

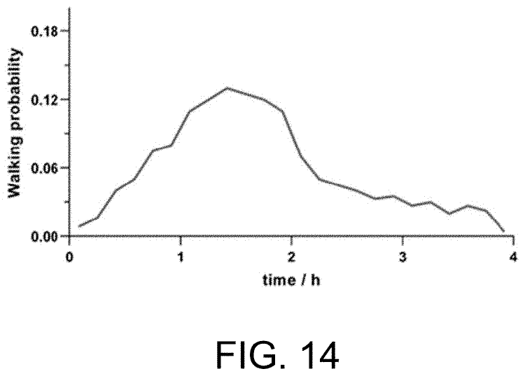

In addition to the foregoing, the program code may build a histogram from the occurrences that shows the walking probability for the patient throughout the day, which may be provided to the clinician and/or patient in one or more reports, which may be provided periodically or on-demand. FIG. 13 illustrates walking probability as a function of time on the basis of data in the patient profile, as well as vertical bars that correspond to times as which the patient was administered medication. As such, the system may provide the clinician with a visualization of the relationship between motor functionality and medication.

Where sufficient gait events that have been collected over the time window create a daily profile that is patient specific, step 806, program code calculates a walking probability profile with a given medication window, step 808. Such data may then be split in accordance with a plurality of medication windows that can be merged together to obtain a medication window profile. According to one or more embodiments, a given medication window is four (4) hours in duration, thereby yielding a curve such as FIG. 14 presents.

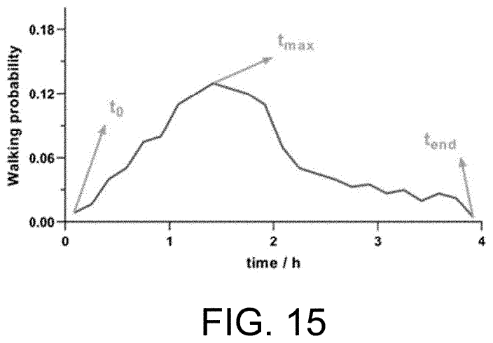

On the basis of data comprising the medication window profile, the program code calculates deltas from t₀, tₘₐₓ, and t_{end}. t₀, tₘₐₓ and t_{end}, step 810, which may be defined in accordance with the data points that FIG. 15 illustrates.

According to the embodiment of FIG. 8, the following condition must be satisfied to calculate a final motor fluctuation score:

tₘₐₓ>t₀˜t_{end}

whereby the outcome of the process is a motor fluctuation score that may be expressed as tₘₐₓ-t₀.

The derived motor fluctuation score may be continuously evaluated, with the motor fluctuation score also output or updated on a continuous or periodic basis, step 812. Program code may provide the motor fluctuation score or value to the patient on his or her mobile device, as well as to the clinician on his or her clinician device. Program code at the cloud-based services may periodically generate reports that include the motor fluctuation score as one among several data points in a given report.

Additional aspects of the present disclosure are further described below in connection with assessing two mobility-related disease events: freezing of gait (“FoG”) and tremor. One or more implementations of the present disclosure include technology for mobile-based continuous assessment of FoG and tremor, respectively.

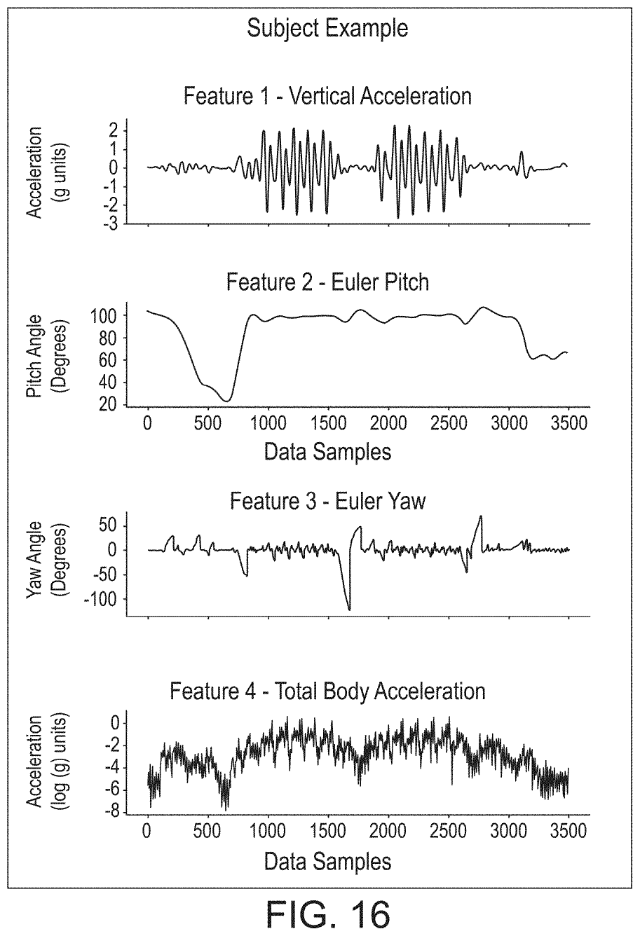

FIGS. 16-21B regard mobile-based continuous assessment, including of FoG (or non-freezing of gait (“noFoG”), in accordance with one or more implementations of the present disclosure. Input data representing walking events, for example, can be collected in a data pipeline and processed as a nearly continuous flow of ecologically assessed gait metrics. The data pipeline can be prepared to collect inertial data from any sensors that are present on a mobile device of the user including, but not limited to, accelerometer, gyroscope and magnetometer. The raw inertial data, for example, including information received from an accelerometer, a gyroscope, and a magnetometer, are received and respective temporal features extracted therefrom.

In addition or in the alternatively, in one or more implementations a continuous flow of ecologically assessed global positioning system (“GPS”) data positions can be received and processed. All GPS points can be converted into distances to the first GPS point of each day, in accordance with the formula set forth below, which provides for improved privacy by making the original geographical location information impossible to determine or recover. In one or more implementations, the process is performed on a smartphone or other mobile computing device, the GPS coordinates are deleted, and only the distances are transferred to the back-end.

$$d = 2 r {sin}^{- 1} ( \sqrt{{sin}^{2} ( \frac{{Φ}_{2} - {Φ}_{1}}{2} ) + cos ( {Φ}_{1} ) cos ( {Φ}_{2} ) {sin}^{2} ( \frac{{λ}_{2} - {λ}_{1}}{2} )} )$$

where d is the distance between the initial position and a new recorded one, Φ₁, Φ₂ represent the latitude of the two positions, and λ₁, λ₂ represent the longitude of the two positions, respectively.

Moreover, a distance profile at different time scales can be provided in accordance with the teachings herein. For example, two probability density profiles can be built based on short (ST) and long (LT) timescales. The ST profile can be built using, for example, all data points from a previous period of time, such as the previous 14 days. The LT profile can be built using, for example, all data points from a longer period of time, such as the previous 90 days. Below are an example ST profile and LT profile:

Using the ST curve and LT curve, such as shown above, a difference between the ST and LT curves can be calculated and a new curve is obtained, such as shown below.

Participation momentum can be determined, for example by using the curve shown above, and to calculate a participation score (PM) given by:

$${P}_{M} = {\sum }_{d = 0} [ S T ( d ) - L T ( d ) ] \frac{1}{1 + {e}^{- A d}}$$

Using this analysis, clinicians can expand on patients' trend-following momentum of the patients' participation and can signal important acute changes in respective participation patterns concerning patients' long-term patterns. Numerous analyses can be dissected; for example, a crossover between patterns can indicate a reversal, for example, either positive (up) or negative (down). Upon recognizing such condition, one or more alert or action can be directed to be taken by a healthcare team.

Example representation of participation momentum is provided below.

The relevance of the participation momentum is given by the participation oscillator, such as shown below, where light green, dark green, light red and dark red indicate values observed in a patient's long-term pattern in different cut-off percentages based on his/her history, respectively. This analysis opens the possibility to flag or other trigger specific actions to reinforce stimulus, following positive behavior (light or dark green) or to identify potential red flag short-term behaviors (light and dark red) that, once accumulated, can convert to long-term unwanted patterns.

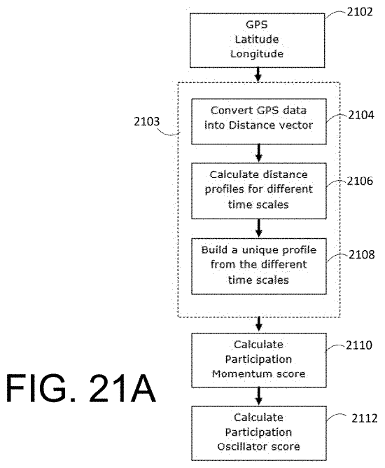

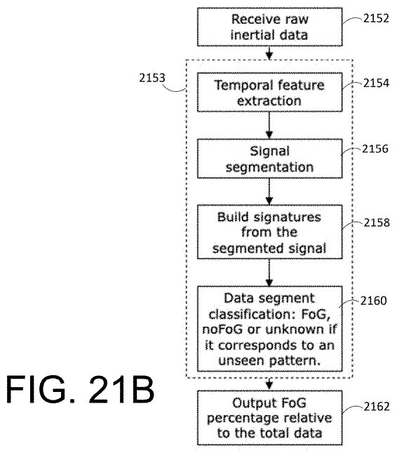

FIG. 21A presents a flow drawing that illustrates steps in an example process for mobile-based continuous assessment of participation, according to one or more embodiments of the present disclosure. At step 2102, GPS latitude and longitude data are received. Steps 2104-2108 collectively are comprised in subprocess 2103, which includes processing the received GPS data to generate a unique profile from the different time scales.

Continuing with reference to subprocess 2103 shown in the flow drawing in FIG. 21A, at step 2104 the GPS data received during step 2102 are processed by one or more computing devices and converted into a distance vector. At step 2106, distance profiles at different time scales are calculated. Thereafter, at step 2108, a unique profile from the different time scales is generated. At step 2110, a participation momentum score is calculated and, thereafter, at step 2112, a participation oscillator score is calculated. Thereafter, the process ends.

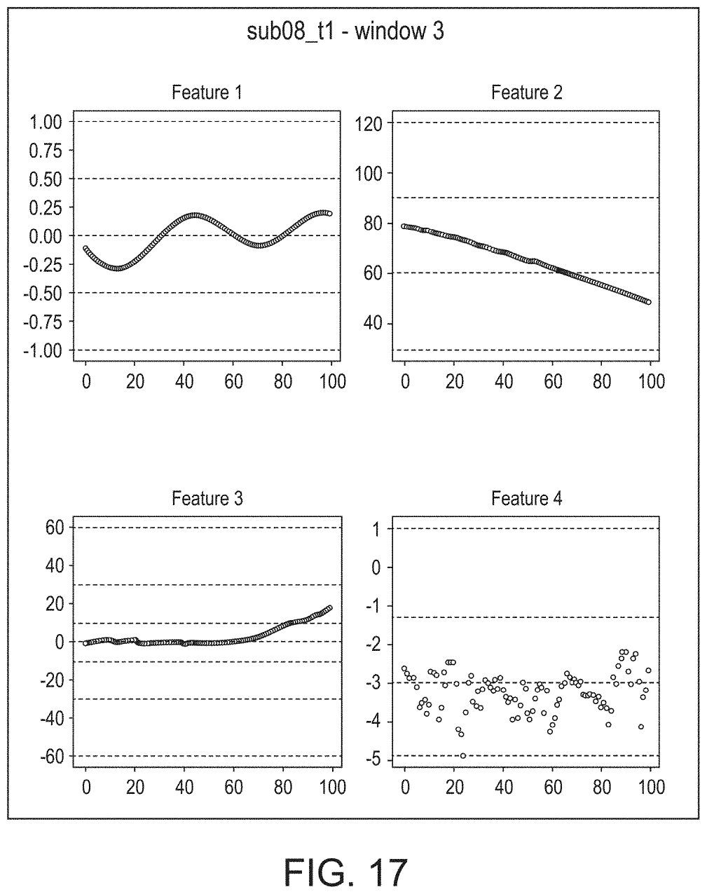

As noted above and according to one or more embodiments, temporal features of a given event can include the beginning or duration of a gait event. The process may utilize stride time or length, cadence, or other feature, as opposed to or in conjunction with gait metric features. Example line graphs representing extracted features, such as shown and described herein from the raw inertial data are shown in FIG. 16. As shown in FIG. 16, Feature 1 represents vertical acceleration, Feature 2 represents Euler pitch, Feature 3 represents Euler yaw, and Feature 4 illustrates total body acceleration. Further, the obtained raw data signal can be divided (or split) into short segments, such as one second segments, and graphed into a list of blocks, which are usable to classify respective data as representing FoG or noFoG. FIG. 17 illustrates four example graphs, each representing a segmented signal in connection with respective ones of four extracted features from the raw inertial data shown and described with reference to FIG. 16.

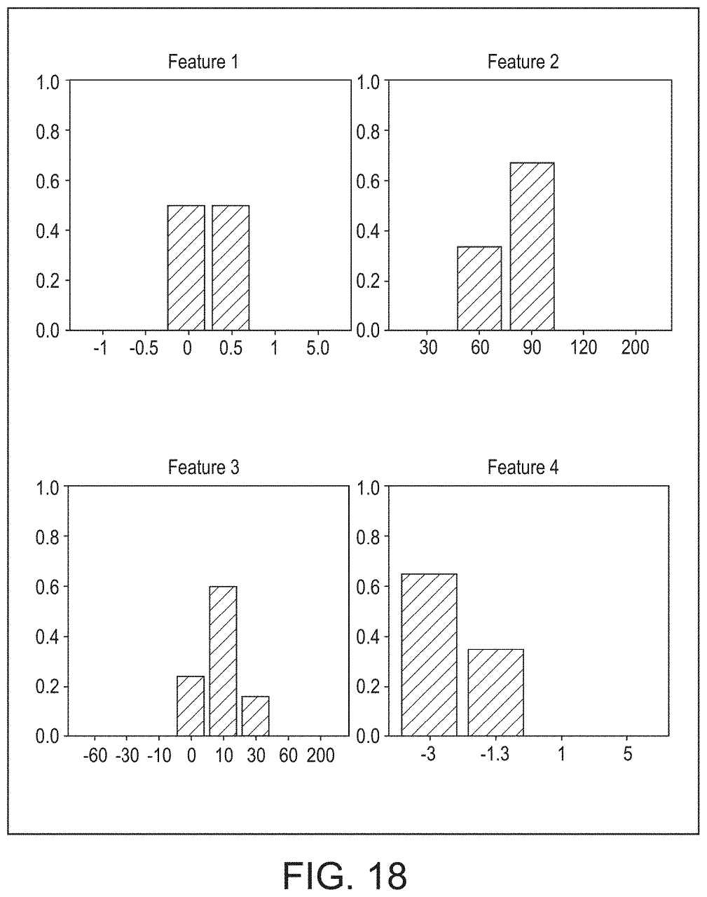

In one or more implementations of the present disclosure, the obtained segments for each of the respective features extracted from the raw inertial data (e.g., as represented FIG. 17) are discretized. For example, the obtained segments can be discretized in accordance with corresponding dashed lines shown in each graph, respectively associated with Features 1-4 in FIG. 17. One or more processors can process the discretization to generate a respective histogram for each of the features (e.g., Features 1-4). As shown in FIG. 17, the histograms correspond to respective signatures generated for a segment, such as illustrated in FIG. 18.

Continuing with reference to FIG. 18, one or more processes can be configured to process the obtained signatures (e.g., as represented in FIG. 18), such as by comparing the obtained signatures with other signatures stored in one or more databases. Such stored signatures can have been obtained during previous clustering of gait segments and, thereafter, identified FoG or noFoG. According to the distance to each cluster, a new point can be classified as FoG, noFoG, or unknown, for example, in case a new point corresponds to a previously unseen pattern.

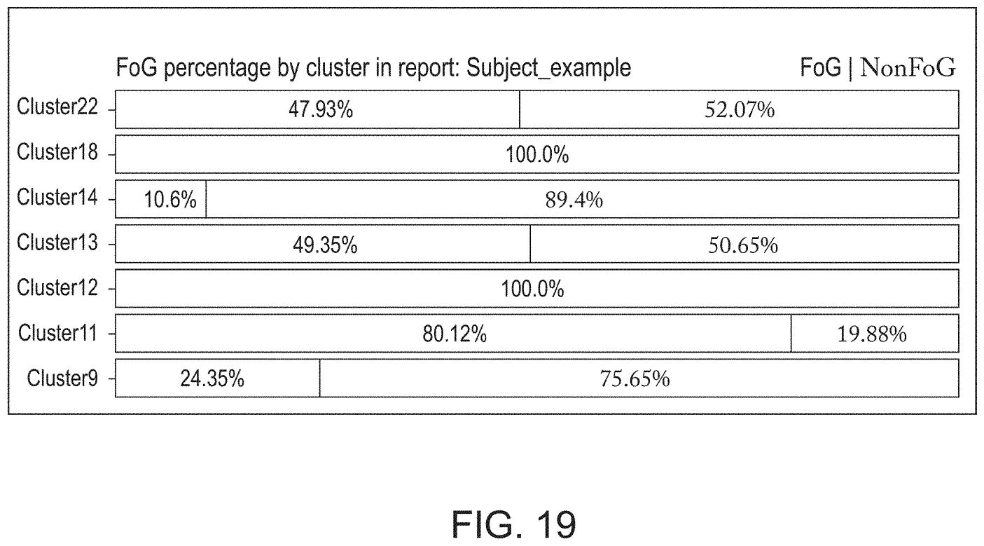

FIG. 19 illustrates an example report that identifies FoG and nonFoG percentages for a respective cluster.

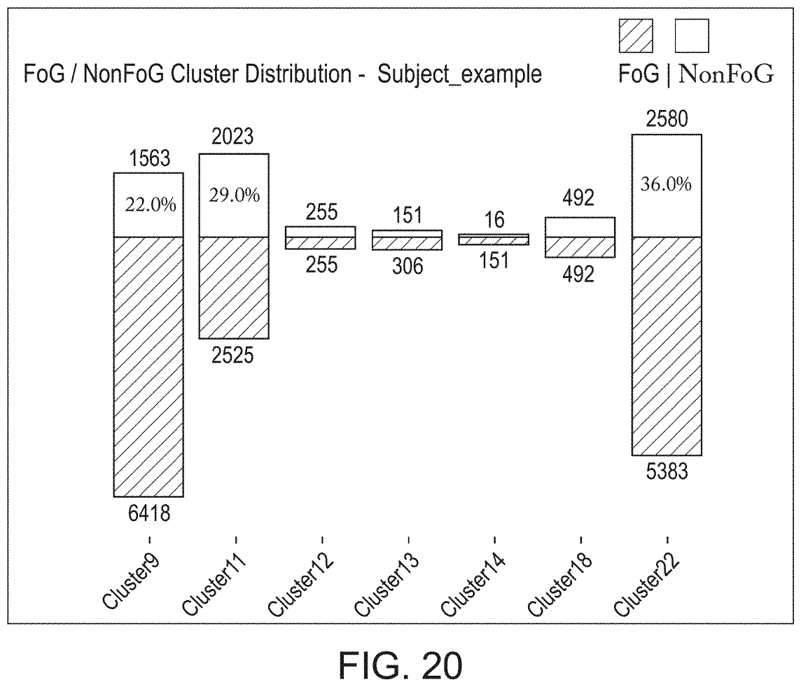

FIG. 20 illustrates an example graph, which shows a distribution of FoG/nonFoG clusters in an example subject. As shown in FIG. 20, values below and above the respective bars represent a number of blocks corresponding to each condition. Further, the respective percentages represent the fraction of each FoG cluster on the total FoG labels, for the given example.

FIG. 21B presents a flow drawing that illustrates steps in an example process for mobile-based continuous assessment of FoG, according to one or more embodiments of the present disclosure. At step 2152, raw inertial data are received. For example, raw inertial data can include information received from one or more sensors on a mobile device of a subject, and which can be provided as inputs for assessing FoG in accordance with one or more implementations of the present disclosure. Steps 2154-2160 collectively are comprised in subprocess 2153, which includes processing the received raw inertial data to generate FoG and nonFoG percentages for each of a plurality of respective clusters, such as shown and described with reference to FIG. 20.

Continuing with reference to subprocess 2153 shown in the flow drawing in FIG. 21B, at step 2154 raw inertial data received during step 2152 are processed by one or more computing devices for temporal feature extraction. For example, temporal features of a given gait event, e.g., the beginning and the duration of the gait event, can be identified. According to one or more implementations of the present disclosure, information such as stride time or length, and cadence can be extracted. At step 2156, one or more computing devices perform signal segmentation, such as to split a signal into short segments, such as one second segments that can be graphed into a list of blocks and usable to classify respective data, for example, as representing FoG. At step 2158, the one or more computing devices can process the segmented signal to build signatures therefrom. The multidimensional blocks can be then clustered, for example, using an Affinity Propagation Algorithm guided by a stability cluster validation index. Unsupervised clustering can then be driven by the unlabeled data, with the result of the procedure being used as classifier labels. This method allows for generating a continuous and unbiased classification of movement states. Furthermore, this methodology allows for access to the microstructure of continuous movement and sequences and quantify transitions and movement variability, both in long-term monitoring and also in more short-term movement analysis.

Continuing with reference to subprocess 2153 shown in the flow drawing in FIG. 21B, at step 2160 one or more computing devices operate to classify each respective data segment as FoG or noFoG. For example, signatures can be obtained and compared with stored signatures in one or more databases that are identified as FoG or noFoG. In one or more implementations, the stored signatures were previously obtained during clustering of respective gait segments and, thereafter, stored and made accessible via cloud-based services 210. During processing, an unrecognized or previously unseen signature pattern can be identified, for example, as unknown. In one or implementations of the present disclosure the distance to each cluster, a new point can be classified as FoG, noFoG, or unknown, for example, in case a new point corresponds to a previously unseen pattern. Thereafter, at step 2162, one or more computing devices output FoG percentages for each of a respective cluster, such as represented in a bar graph of blocks corresponding to a respective condition. Output can further include percentages representing fractions of each respective FoG cluster on the total FoG labels, in a given instance. Thereafter, the process ends.

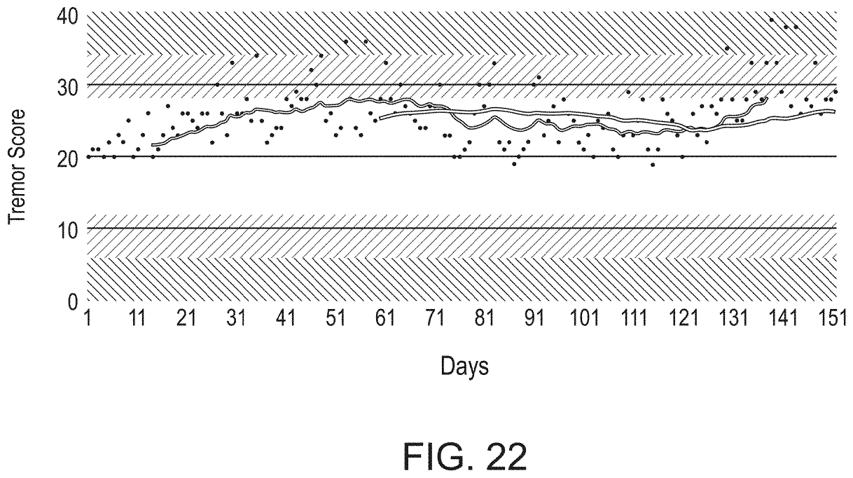

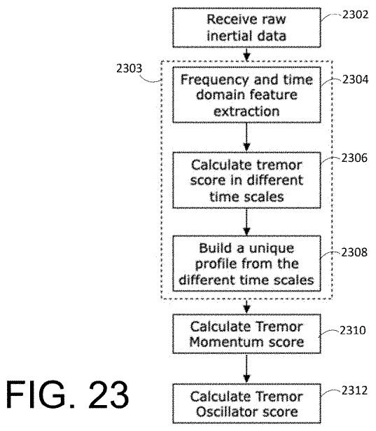

FIGS. 22 and 23 regard mobile-based continuous tremor assessment, in accordance with one or more implementations of the present disclosure. Input data are provided using inertial data recorded during a given period, for example, 5 seconds, as a computing device is handled by a user. Information representing possible tremor events can be collected, in one or more implementations, in a data pipeline configured to collect inertial data from sensors that are present on the mobile device of the user including, but not limited to, accelerometer, gyroscope and magnetometer. One or more computing devices are configured to process the raw inertial data, including by defining blocks of a respective sample size for frequency analysis.

For example, several frequency and time domain features can be extracted from raw inertial data, including power spectrum, dominant frequency, root mean square (RMS), vertical acceleration, Euler angles or total body acceleration.

In one or more implementations, one or more computing devices can filter the frequency domain of an obtained signal for use to compute a Tremor score. The Tremor score value can be a ratio of the spectral range of interest, e.g., 3 and 7 Hz in Parkinson's disease, and the normalized spectral median power. Thereafter, the ratio can be scaled by the same range's root mean square. An example equation for calculating a Tremor score can be as follows:

$$Tr Score ≃ \frac{{\sum }_{i}^{n} abs ( {Freq}_{i} )}{{\sum }_{c}^{fs / 2} Px} * \sqrt{{\sum }_{i}^{n} \frac{1}{( n - i + 1 )} F r e {q}_{i}^{2}}$$

where Freq represents the magnitude of each frequency component, fs the frequency of the sample and Px is the power of each frequency component. The range of interest is i and n, respectively.

Using the Tremor score, tremor momentum can be determined, in the form of a Tremor Momentum score. For example, a list of Tremor scores can be calculated for each data point collected, and information representing a date and/or time is output. Thereafter, one or more computing devices can calculate two exponential moving averages, e.g., Short and Long, based on respective Tremor scores as well as, for example, one or more previous timeframes (e.g., 14 days and 90 days, respectively). Similar to determining tremor momentum, clinicians can expand on patient analysis as a function of trend-following momentum of a patient's short-term tremor pattern changes with respect to patient's long-term patterns. Numerous analyses can be dissected, for example, to indicate a reversal as a function of a crossover between patterns, including positive (up) or negative (down). Upon recognizing such condition, one or more alert or action can be directed to be taken by a healthcare team.

The relevance of Tremor Momentum is represented by a tremor oscillator. For example, light green, dark green, light red and dark red indicate values that were observed in different cut-off percentages, based on a patient's history (for example, +68% and +95%). Similar to participation oscillator, the ability to flag/trigger specific actions to reinforcing stimulus following a positive behavior (light or dark green) or to identify potential red flag short-term behaviors (light and dark red) that once accumulated can convert to long-term unwanted patterns, can be realized.

FIG. 22 is an example report representing a tremor oscillator, which illustrates a graph representing calculated Tremor scores over a course of time (e.g., days).

FIG. 23 presents a flow drawing that illustrates steps in an example process for mobile-based continuous tremor assessment, according to one or more embodiments of the present disclosure. At step 2302, raw inertial data are received. For example, raw inertial data can include information received from one or more sensors on a mobile device of a subject, and which can be provided as inputs for tremor assessment, in accordance with one or more implementations of the present disclosure. Steps 2304-2308 collectively are comprised in subprocess 2303, which includes processing the received raw inertial data. At step 2304, one or more computing devices perform frequency and time domain frequency extraction. Thereafter, at step 2306, one or more computing devices calculate a tremor score in different time scales. Using output from step 2306, a unique profile is built from the respective time scales (step 2308). Thereafter, one or more computing devices calculates a Tremor Motion score. At step 2312, a Tremor Oscillator score is calculated. Thereafter, the process ends. The tremor oscillator can be considered a frequency distribution of the Momentum scores obtained over time plotted with specific cut-off values—for example, 1× or 2× the data standard deviation corresponding to +68% and +95% of the amount of historical data variation.

The drawings set forth herein are conceptual illustrations allowing for an explanation of the present invention. Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

The foregoing description of the specific embodiments will so fully reveal the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present invention have been described above, it should be understood that they have been presented by way of example, and not limitation. It would be apparent to one skilled in the relevant art(s) that various changes in form and detail could be made therein without departing from the spirit and scope of the invention. Thus, the present invention should not be limited by any of the above-described exemplary embodiments, but rather should be defined only in accordance with the following claims and their equivalents.

What is claimed:

1. A computer-implemented mobility monitoring method, the method comprising:
receiving by at least one computing device configured by executing instructions stored on processor-readable media, inertial data captured by at least one sensor configured with a mobile computing device, the inertial data representing at least one aspect of a subject's orientation in three-dimensional space;
processing, by the at least one computing device, the inertial data to normalize the inertial data, the normalized inertial data representing a change in the subject's orientation, wherein processing the inertial data includes:
integrating at least one gyroscope measurement representing attitude with at least one accelerometer measurement representing the distance to compensate for long-term gyroscope integration drift and to obtain a global orientation of the mobile computing device; and
applying a rotation matrix to calculate a vertical component of acceleration of the mobile computing device;
determining, by the at least one computing device using the normalized inertial data, gait patterns associated with the subject, wherein determining the gait patterns includes parameters including at least one of:
assessing a stride duration by calculating a time difference between two ipsilateral heel-strike events;
assessing a stance phase duration by calculating a time difference between a heel-strike event and an ipsilateral toe-off event that occurred after the heel-strike event;
assessing a swing phase duration by calculating a time difference between a toe-off event and an ipsilateral heel-strike event that occurred after the toe-off event;
assessing a stride time by calculating a number of strides per minute;
assessing a stride length by calculating a linear relation with stride frequency; and
assessing an acceleration variance by dividing the stride length by the stride time;
classifying, by the at least one computing device using the parameters of the determined gait patterns, the subject's gait;
generating, by the at least one computing device, information associated with the subject's classified gait; and
transmitting, by the at least one computing device to at least one other computing device, the generated information associated with the subject's classified gait.

2. The computer-implemented mobility monitoring method of claim 1, wherein the at least one of the ipsilateral heel-strike event, the heel-strike event, the ipsilateral toe-off event is detected by at least one mobile computing device using a peak detection algorithm.

3. The computer-implemented mobility monitoring method of claim 1, further comprising:
processing, by the at least one computing device, information associated with results of the peak detection algorithm, wherein the processing of the information includes at least one of:
smoothing, by the at least one computing device, a detected noisy acceleration signal;
attenuating differences between information received from at least one mobile computing device positioned differently on the subject;
detecting toe-off events by identifying peaks in a first derivative of a received acceleration signal;
disregarding information representing a stride that is smaller by a predetermined threshold than a previous stride and a next stride; and
identifying and removing anomalous events as a function of outlier detection by having an isolation forest model applied to at least some received information.

4. The computer-implemented mobility monitoring method of claim 3, further comprising:
discarding, by the at least one computing device, at least one gait event having a frequency above a pre-determined threshold.

5. The computer-implemented mobility monitoring method of claim 1, further comprising:
receiving, by the at least one computing device from at least one mobile computing device, location data representing a plurality of positions at respective times; and
processing, by the at least one computing device, the location data to generate distance information representing distances from a first location position represented in the location data to at least one other location position represented in the location data.

6. The computer-implemented mobility monitoring method of claim 5, further comprising:
generating, by the at least one computing device using at least some of the location data, a first distance profile associated with at least some of the location data representing respective location positions at a first set of respective times, and a second distance profile associated with at least some of the location data representing location positions at a second set of respective times,
wherein the second set of respective times is longer than the first set of respective times.

7. The computer-implemented mobility monitoring method of claim 6, further comprising:
determining, by the at least one computing device using information associated with the first profile and the second profile, participation momentum; and
calculating, by the at least one computing device as a function of the participation momentum, a participation score.

8. The computer-implemented mobility monitoring method of claim 1, further comprising:
identifying, by the at least one computing device using an oscillator, at least one of positive behavior and negative behavior that can impact long-term behavior patterns.

9. The computer-implemented mobility monitoring method of claim 1, further comprising:
synchronizing, by the at least one computing device, orientation data from at least one sensor using linear interpolation and spherical linear interpolation; and
performing, by the at least one computing device using a Madgwick Orientation Filter, sensor fusion.

10. The computer-implemented mobility monitoring method of claim 1, further comprising:
receiving, in response to a prompt provided on a mobile computing device, testing information associated with remote active capacity testing.

11. The computer-implemented mobility monitoring method of claim 10, wherein the testing information includes results of at least one of a balance test, a finger tapping exercise, and a walk test.

12. A computer-implemented mobility monitoring system, the system comprising:
at least one computing device configured by executing instructions stored on processor-readable media to perform operations, including:
receiving, by at least one computing device configured by executing instructions stored on processor-readable media, inertial data captured by at least one sensor configured with a mobile computing device, the inertial data representing at least one aspect of a subject's orientation in three-dimensional space; and
processing, by the at least one computing device, the inertial data to normalize the inertial data, the normalized inertial data representing a change in the subject's orientation, wherein processing the inertial data includes:
integrating at least one gyroscope measurement representing attitude with at least one accelerometer measurement representing distance to compensate for long term gyroscope integration drift and to obtain a global orientation of the mobile computing device; and
applying a rotation matrix to calculate a vertical component of acceleration of the mobile computing device;
determining, by the at least one computing device using the normalized inertial data, gait patterns associated with the subject, wherein determining the gait patterns includes parameters including at least one of:
assessing a stride duration by calculating a time difference between two ipsilateral heel-strike events;
assessing a stance phase duration by calculating a time difference between a heel-strike event and an ipsilateral toe-off event that occurred after the heel-strike event;
assessing a swing phase duration by calculating a time difference between a toe-off event and an ipsilateral heel-strike event that occurred after the toe-off event;
assessing a stride time by calculating a number of strides per minute;
assessing a stride length by calculating a linear relation with stride frequency; and
assessing an acceleration variance by dividing the stride length by the stride time;
classifying, by the at least one computing device using the parameters of the determined gait patterns, the subject's gait;
generating, by the at least one computing device, information associated with the subject's classified gait; and
transmitting, by the at least one computing device to at least one other computing device, the generated information associated with the subject's classified gait.

13. The computer-implemented mobility monitoring system of claim 12, wherein at least one of the ipsilateral heel-strike event, the heel-strike event, the ipsilateral toe-off event is detected by at least one mobile computing device using a peak detection algorithm.

14. The computer-implemented mobility monitoring system of claim 12, wherein the at least one computing device is further configured by executing instructions stored on processor-readable media to perform operations, including:
processing, by the at least one computing device, information associated with results of the peak detection algorithm, wherein the processing the information includes at least one of:
smoothing, by the at least one computing device, a detected noisy acceleration signal;
attenuating differences between information received from at least one mobile computing device positioned differently on the subject;
detecting toe off events by identifying peaks in a first derivative of a received acceleration signal;
disregarding information representing a stride that is smaller by a predetermined threshold than a previous stride and a next stride; and
identifying and removing anomalous events as a function of outlier detection having an isolation forest model applied to at least some at least some received information.

15. The computer-implemented mobility monitoring system of claim 14, wherein the at least one computing device is further configured by executing instructions stored on processor-readable media to perform operations, including:
discarding, by the at least one computing device, at least one gait event having a frequency above a pre-determined threshold.

16. The computer-implemented mobility monitoring system of claim 12, wherein the at least one computing device is further configured by executing instructions stored on processor-readable media to perform operations, including:
receiving, by the at least one computing device from at least one mobile computing device, location data representing a plurality of positions at respective times; and
processing, by the at least one computing device, the location data to generate distance information representing distances from a first location position represented in the location data to at least one other location position represented in the location data.

17. The computer-implemented mobility monitoring system of claim 16, wherein the at least one computing device is further configured by executing instructions stored on processor-readable media to perform operations, including:
generating, by the at least one computing device using at least some of the location data, a first distance profile associated with at least some of the location data representing respective location positions at a first set of respective times, and a second distance profile associated with at least some of the location data representing location positions at a second set of respective times,
wherein the second set of respective times is longer than the first set of respective times.

18. The computer-implemented mobility monitoring system of claim 17, wherein the at least one computing device is further configured by executing instructions stored on processor-readable media to perform operations, including:
determining, by the at least one computing device using information associated with the first profile and the second profile, participation momentum; and
calculating, by the at least one computing device as a function of the participation momentum, a participation score.

19. The computer-implemented mobility monitoring system of claim 12, wherein the at least one computing device is further configured by executing instructions stored on processor-readable media to perform operations, including:
identifying, by the at least one computing device using an oscillator, at least one of positive behavior and negative behavior that can impact long-term behavior patterns.

20. The computer-implemented mobility monitoring system of claim 12, wherein the at least one computing device is further configured by executing instructions stored on processor-readable media to perform operations, including:
synchronizing, by the at least one computing device, orientation data from at least one sensor using linear interpolation and spherical linear interpolation; and
performing, by the at least one computing device using a Madgwick Orientation Filter, sensor fusion.

21. The computer-implemented mobility monitoring system of claim 12, wherein the at least one computing device is further configured by executing instructions stored on processor-readable media to perform operations, including:
receiving, in response to a prompt provided on a mobile computing device, testing information associated with remote active capacity testing.

22. The computer-implemented mobility monitoring system of claim 21, wherein the testing information includes results of at least one of a balance test, a finger tapping exercise, and a walk test.
